(19) United States
(12) Patent Application Publication
(10) Pub. No.: US 2015/0190600 A1
(43) Pub. Date: Jul. 9, 2015

(54) PATIENT INTERFACE

(71) Applicant: KONINKLIJKIE PHILIPS N.V., EINDHOVEN (NL)

(72) Inventors: Harmina Christina Zeijlstra, Breda (NL); Sander Theodoor Pastoor, Utrecht (NL); Peter Martens, Eindhoven (NL); Antonius Adrianes Petrus Schudelaro, Tilburg (NL); Chrostoph Dobrusskin, Eindhoven (NL); Richard Johannus Maria Van De Ven, Noord Brabant (NL)

(21) Appl. No.: 14/413,242

(22) PCT Filed: Jul. 1, 2013
(86) PCT No.: PCT/IB2013/055375

§ 371 Date: Jan. 7, 2015

Related U.S. Application Data
(60) Provisional application No. 61/670,195, filed on Jul. 11, 2012.

(51) Int. Cl. A61M 16/06; A61M 16/08

(52) U.S. Cl. CPC A61M 16/0644; A61M 16/0622; A61M 16/0683; A61M 16/0816

(57) ABSTRACT

A patient interface comprises a patient interface element for delivering a gas a patient, and a forehead support. An improved adjustment mechanism is provided for the forehead support.

### FIELD OF THE INVENTION

The present invention relates to patient interfaces for transporting a gas to and/or from an airway of a user.

### BACKGROUND OF THE INVENTION

There are numerous situations where it is necessary or desirable to deliver a flow of breathing gas non-invasively to the airway of a patient, i.e. without inserting a tube into the airway of the patient or surgically inserting a tracheal tube in their oesophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnoea syndrome, in particular, obstructive sleep apnoea (OSA).

Non-invasive ventilation and pressure support therapies involve the placement of a patient interface element comprising a mask component on the face of a patient. The mask component may be, without limitation, a nasal mask that covers the patient's nose, a nasal pillow/cushion having nasal prongs that are received within the patient's nostrils, a nasal/oral mask that covers the nose and mouth, or a full face mask that covers the patient's face. The patient interface element interfaces between the ventilator or pressure support device and the airway of the patient, so that a flow of breathing gas can be delivered from the pressure/flow generating device to the airway of the patient.

Such elements are typically maintained on the face of a patient by headgear having one or more straps adapted to fit over/around the patient's head.

FIG. 1 shows a typical system to provide respiratory therapy to a patient. This system will be referred to in the description and claims as a “patient interface assembly”. The assembly 2 includes a pressure generating device 4, a delivery conduit 16 coupled to an elbow connector 18, and a patient interface 10. The pressure generating device 4 is structured to generate a flow of breathing gas and may include, without limitation, ventilators, constant pressure support devices (such as a continuous positive airway pressure device, or CPAP device), variable pressure devices, and auto-titration pressure support devices.

Delivery conduit 16 communicates the flow of breathing gas from pressure generating device 4 to patient interface 10 through the elbow connector 18. The delivery conduit 16, elbow connector 18 and patient interface device 10 are often collectively referred to as a patient circuit.

The patient interface 10 includes a patient interface element which is a mask 12 in the form of a shell 15 and cushion 14, which in the exemplary embodiment is a nasal and oral mask. However, any type of mask, such as a nasal-only mask, a nasal pillow/cushion or a full face mask, which facilitates the delivery of the flow of breathing gas to the airway of a patient, may be used as mask. The cushion 14 is made of a soft, flexible material, such as, without limitation, silicone, an appropriately soft thermoplastic elastomer, a closed cell foam, or any combination of such materials.

An opening in the shell 15, to which elbow connector 18 is coupled, allows the flow of breathing gas from pressure generating device 4 to be communicated to an interior space defined by the shell 15 and cushion 14, and then to the airway of a patient.

The patient interface assembly also includes a headgear component 19, which in the illustrated embodiment is a two-point headgear. Headgear component 19 includes a first and a second strap 20, each of which is structured to be positioned on the side of the face of the patient above the patient's ear.

Headgear component 19 further includes a first and a second mask attachment element 22 to couple the end of one of the straps 20 to the respective side of mask 12.

A problem with this type of assembly is that the headgear force vectors necessary to achieve a robust and stable seal against the face of the patient can cut a straight line near the corners of a patient's eyes, which can be uncomfortable and distracting.

In order to avoid this, it is well known to include as part of the patient interface a forehead support to spread the required forces over a larger area. In this way, an additional cushion support on the forehead balances the forces put by the mask around the nose or nose and mouth.

All faces are different to each other. When using a patient interface assembly which has a forehead support, this forehead support should be adjustable for personal fit. The offset between facial plane and the forehead support can differ in the range of 30 mm.

An example of known adjustment arrangement uses a rotating mechanism, controlled by a rotary knob. This mechanism results in an increase or decrease of the offset between the facial plane and the forehead support. The user has to rotate the knob to get the right offset and the right fit.

From an ergonomics perspective, rotating a knob is not the most convenient way to implement adjustment. The number of elements and required accuracy is costly and the assembly can be noise due to play between components.

### SUMMARY OF THE INVENTION

According to the invention, there is provided a patient interface as claimed in the independent claims.

In one aspect, the invention provides a patient interface comprising:

a patient interface element for delivering a breathing gas to a patient; and

a forehead support coupled to the patient interface and having a support cushion,

wherein one of the forehead support and the patient interface element comprises a shaft and the other of the forehead support and the patient interface element comprises an opening in which the shaft is received, with the shaft slidable within the opening to permit adjustment of the position of the forehead support relative to the patient interface element, wherein locking means is provided for fixing the position of the shaft and opening, and a release button is provided for releasing the locking means.

This arrangement enables simple sliding adjustment of the position of the forehead support, with the forehead support then locked in the desired position.

In a first version, the shaft is attached to the forehead support, and the patient interface element comprises a frame part which comprises:

a housing through which the shaft passes;

a drive spring in the housing;

a drive member for driving the shaft having a drive configuration in which it grips the shaft and a release configuration in which it releases the shaft, wherein the drive member is biased by the drive spring to the release configuration;

the locking means comprises a locking member for locking the shaft having a locked configuration in which it grips the shaft and a release configuration in which it releases the shaft, wherein the locking member is biased by a locking spring to the locked configuration.

This arrangement defines a spring loaded pump arrangement. Each advance of the shaft is driven by the drive member (with the locking member in a release configuration) and then the new shaft position is held by the locking member, with the drive member in its released sate.

The drive member can comprise a pivoted plate having an opening through which the shaft passes and which is biased by the drive spring into an orientation perpendicular to the shaft axis. This perpendicular orientation is the released configuration, and the opening has a shape matched to the shaft cross section.

The locking member can also comprise a pivoted plate having an opening through which the shaft passes and which is biased by the locking spring into an orientation offset from perpendicular to the shaft axis, i.e. biased into the locked configuration.

In another version, the shaft is attached to the patient interface element (for example to the frame part), and the opening is formed in a tube attached to the forehead support, wherein the locking means comprises a frictional pad which is coupled to the shaft by a spring mechanism and is biased against the inner surface of the tube, wherein the release button is for depressing the frictional pad against the bias of the spring mechanism.

This arrangement enables sliding adjustment with a frictional brake. The tube can comprise a slot through which the release button extends. This defines the range of adjustment.

Another aspect of the invention provides a patient interface comprising:

a patient interface element for delivering a breathing gas to a patient; and

a forehead support coupled to the patient interface and having a support cushion,

wherein the forehead support comprises a forehead cam arrangement and the patient interface element comprises a patient interface cam arrangement, wherein the relative positioning of the patient interface and forehead cam arrangements in a first plane implements adjustment of the position of the forehead support along an axis perpendicular to the first plane.

This arrangement uses adjustment of cams to provide the forehead support height adjustment. The adjustment is perpendicular to the forehead support adjustment direction, so that simple lateral adjustment can be made.

An elastic connector can be used to couple the cam arrangements together.

The forehead cam arrangement and the patient interface cam arrangement can each comprise two side by side cam blocks, wherein the patient interface cam arrangement blocks are slideable in the first plane relative to the patient interface element, wherein the position adjustment is effecting by moving the patient interface cam arrangement blocks together or apart. The cam arrangements can each comprise a stepped cam surface to define a set of discrete adjustment locations, or else smooth cam surfaces can be used.

### BRIEF DESCRIPTION OF THE DRAWINGS

Examples of the invention will now be described in detail with reference to the accompanying drawings, in which:

FIG. 1 shows a known patient interface;

FIG. 2 shows a known patient interface as disclosed in US2010/0000542;

FIG. 3 shows a first example of patient interface of the invention;

FIG. 4 shows two control settings of the patient interface of FIG. 3;

FIG. 5 shows a second example of patient interface of the invention.

FIG. 6 shows a third example of patient interface of the invention; and

FIG. 7 shows the patient interface of FIG. 6 in more detail.

The invention provides a patient interface comprising a patient interface element (i.e. a mask) and a forehead support.

### DETAILED DESCRIPTION OF THE EMBODIMENTS

The invention provides an improved adjustment mechanism for the forehead support.

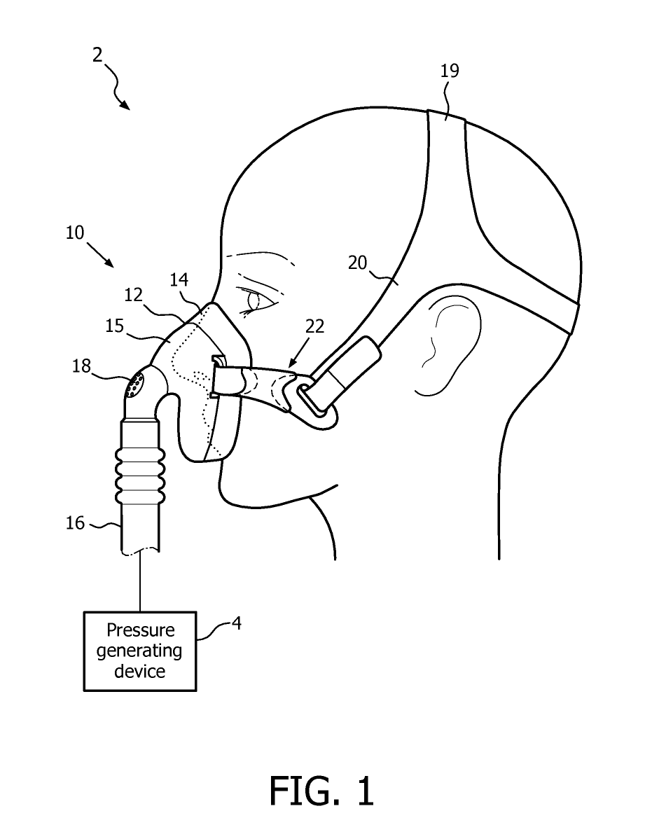

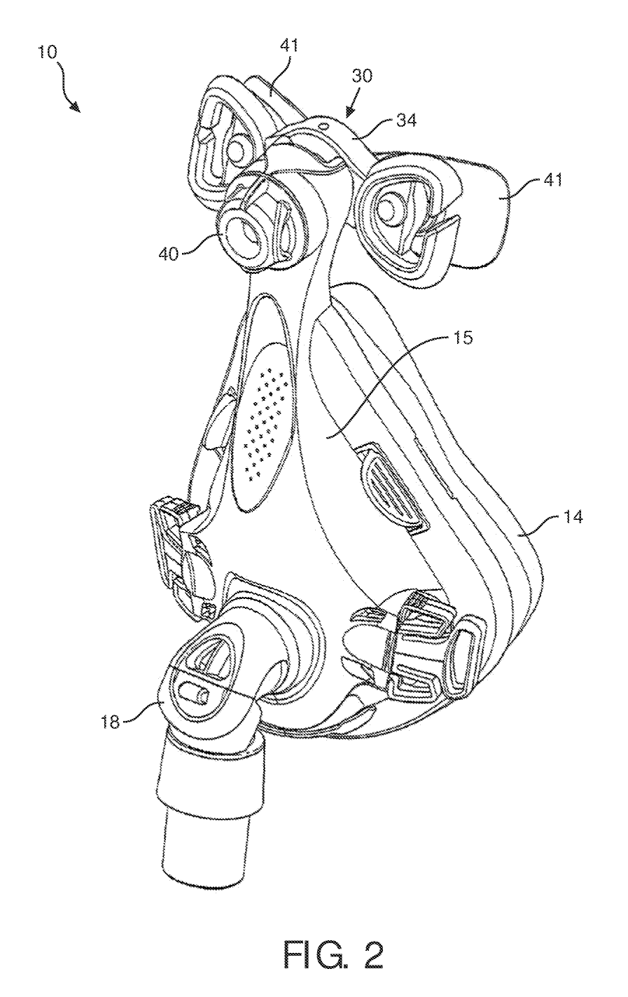

FIG. 2 is taken from US2010/0000542 and shows a patient interface assembly in the form of a full facial mask assembly 10 including a forehead support 30. The patient interface (the mask part) is for delivering gas to the user and includes a frame 16, a cushion 14 adapted to form a seal with the patient's face, an elbow assembly 18 for connection to an air delivery tube (components 10,14,16,18 corresponding to those of the same number in FIG. 1).

FIG. 2 shows a forehead support 30 for reducing the forces on the patient's face, and including a frame 34 which carries forehead support cushions 41. In this example, the position of the forehead support is adjustable by a rotary knob 40.

The rotary knob comprises a screw-type actuator which moves the forehead support 30 along a generally linear path. The rotary adjustment knob 40 includes a threaded shaft, and the forehead support frame 34 includes an internally threaded tube.

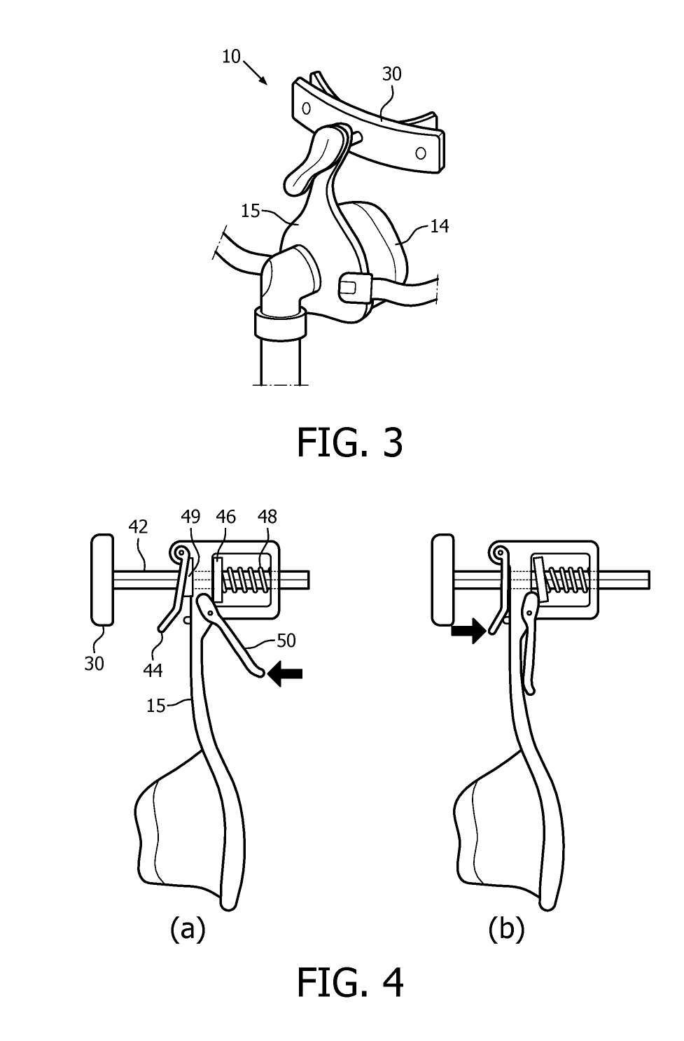

When the adjustment knob 40 is rotated, the internally threaded tube of the forehead support 34 extends or retracts from the threaded shaft of the adjustment knob 40, which causes adjustable movement of the forehead cushions 41. FIG. 3 shows a first example of patient interface 10 of the invention, similar in structure to the design of FIG. 2, with a cushion 14 and shell 15 and a forehead support 30.

The forehead support is slidable in an out with respect to the shell 15. The sliding is controlled in a pump and lock manner. This means the user operates a trigger to incrementally pump the forehead support into position. The mechanism functions in the same way as a conventional pumped glue gun.

The design is shown in more detail in FIG. 4, in which FIG. 4a shows the assembly during adjustment and FIG. 4b shows the assembly locked into a desired position.

The forehead support 30 has a rod 42 which passes through openings in the mask shell 15. These openings define a housing part within which a drive spring 48 is fitted, positioned around the rod 42. The rod 42 passes through a lock handle 44 and a rod-gripper 46. The lock handle 44 is biased outwardly by a spring 49.

The lock handle and rod gripper work in the same way. They comprise a sheet structure with an opening which closely matches the rod shape. Thus, only when the sheet is perpendicular to the rod can the rod pass freely. As soon as there is a relative angle, the rod is gripped in the opening.

When pressing an adjustment handle 50 the rod-gripper 46 grabs on the rod 42 because it tilts. Once it has tilted to grip the rod, it slides (to the right in FIG. 4a) pushing the rod 42 along. The rod is thus pushed along by the pivoting of the handle 50 against the bias of the drive 48. The rod movement pulls the rod gripper 44 in slightly against the bias of its spring 49 (to the right in FIG. 4a) so that the rod is free to pass through the rod gripper.

When releasing the handle 50 the spring 48 presses the rod gripper 46 back, so that it is reset for another drive operation. The rod is gripped by the lock handle 44 which is pushed by the rod and by its spring 49 into its locking position, offset from the perpendicular direction. Thus, when the rod gripper 46 returns to its original position, it does not drag the rod back with it, because the rod is gripped by the lock handle 44 and because the rod gripper 46 has returned to its perpendicular orientation.

A single press of the handle 50 moves the rod 42 by an incremental amount, and multiple pumps can be used to move the rod and forehead support by the desired amount.

When the user wants to loosen the forehead support 30 the small lock handle 44 is pressed and the rod 42 and the forehead support 30 can be moved freely. The lock handle is being pressed to its perpendicular position and the spring is already pushing the rod gripper 46 to its perpendicular position, so that the rod can be moved.

For the user, this design is simple to operate. The mask is put on and the handle is pushed to adjust the forehead support to the correct position.

In the example shown, the foreahead support is incrementally adjusted back from an extended position. However, it may instead be arranged such that the incremental adjustments pump the forehead support outwardly towards the forehead.

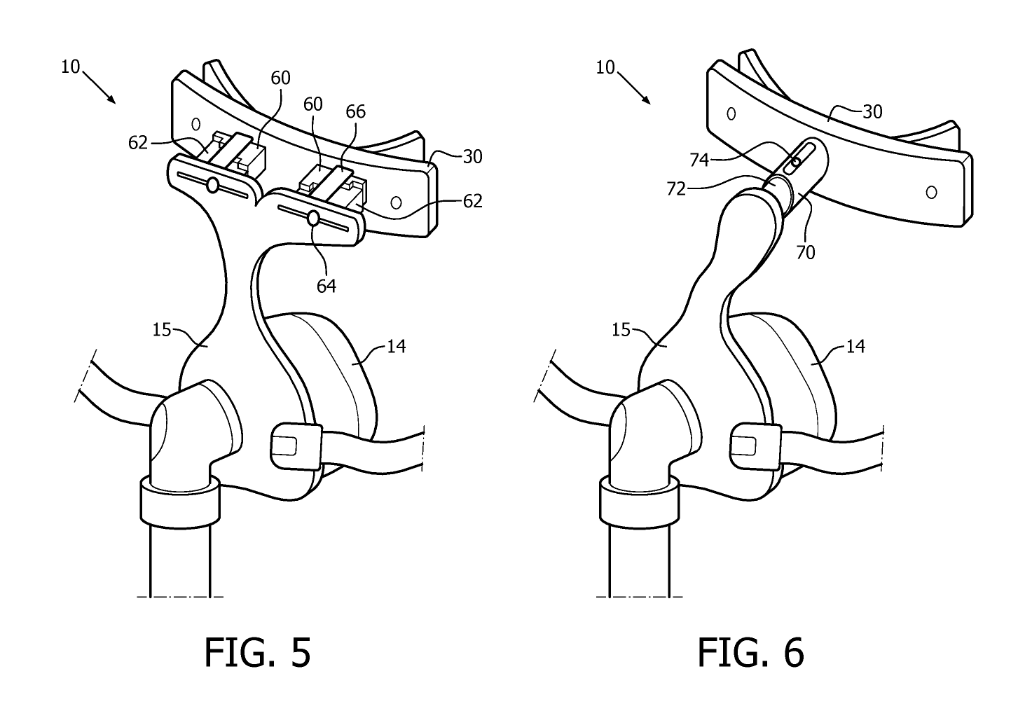

The example above uses linear sliding of the forehead support using a rod connected to the forehead support. FIG. 5 shows a second example of patient interface 10 of the invention, similar in structure to the design of FIG. 2, with a cushion 14 and shell 15 and a forehead support 30.

The shell 15 is connected to the forehead support using cam blocks. The forehead support has two side by side cam blocks 60. They have a cam surface which tapers with respect to the direction of forehead support movement. Thus, one end of the cam surface is nearer the forehead support and the other end is further from the forehead support. The two cam blocks 60 are symmetrically arranged, so that the left-right alignment of the forehead support remains constant during adjustment (as explained below).

The shell 15 has corresponding cam blocks 62. The relative lateral position between a shell cam block 62 and forehead support cam block 60 determines the overall thickness of the combined arrangement. Thus, by setting the lateral spacing between the cam blocks 62, the forehead support distance is adjusted.

In the example shown, the cam surfaces are stepped, so that the adjustment can be considered to be a ratchet function. The cam blocks 62 are connected to the mask shell 15 by a pin 64. The user can move this pin 64 in the mask shell 15 to move the cam block 62 to create a larger or smaller distance between the mask shell 15 and the forehead support 30. The cam block pairs are held to each other by a respective flexible band 66 and these hold the forehead support 30 and the mask shell 15 together.

The cam blocks can have a stepped surface or a flat surface like a wedge. If a smooth surface is provided, the pins 64 can implement a stepped clicking function to make adjustment easier.

This arrangement again provides a simple adjustment mechanism and it uses very few parts.

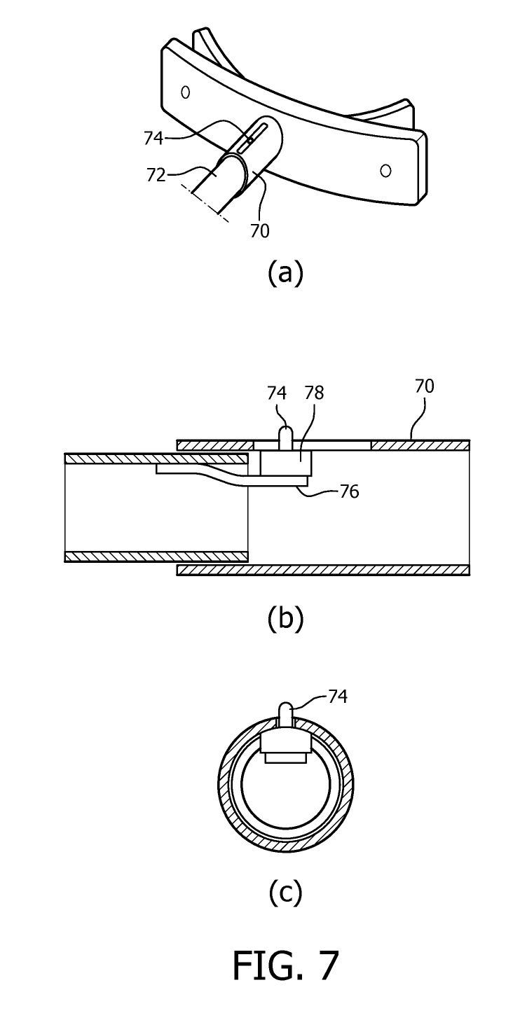

FIG. 6 shows a third example of patient interface 10 of the invention, similar in structure to the design of FIG. 2, with a cushion 14 and shell 15 and a forehead support 30. FIG. 7a shows the control pin more clearly, FIG. 7b shows the friction pad by showing a cross section of the tube and FIG. 8c shows an end view.

This is another sliding arrangement with a frictional retention of the rod in the desired position using a spring loaded friction pad.

A hollow tube 70 is connected to the forehead support 30. This hollow tube is positioned around another adjustment rod 72. The adjustment rod 72 is connected to the mask shell 15. In this way, the adjustment is a sliding of the rod inside the tube. The outer hollow tube 70 has a slot in which a locking button 74 is positioned. The locking button 74 is connected to the inner rod 72. A spring 76 is connected to the inner rod 72 and biased the locking button 76 outwardly. When the user pushes the locking button 74, the system is unlocked and the rod and tube can slide relative to each other. When releasing the locking button 74 the system is locked again.

The locking is achieved by the friction exerted between the base 78 of the locking button and the inside surface of the outer tube 70, created by the spring bias. The base 78 and tube 70 can have surfaces designed to resist sliding, either as rough high friction surfaces or with a series of interlocking engagement notches such as teeth. By using this system the user can adjust the distance of the forehead support in any position with a simple push and slide function.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

1. A patient interface comprising:
a patient interface element for delivering a breathing gas to a patient; and
a forehead support coupled to the patient interface element, wherein one of the forehead support and the patient interface element comprises a shaft and the other of the forehead support and the patient interface element comprises an opening in which the shaft is received, with the shaft slidable within the opening to permit adjustment of the position of the forehead support relative to the patient interface element, and wherein locking means is provided for fixing the position of the shaft and opening, and a release button is provided for releasing the locking means.

2. A patient interface as claimed in claim 1, wherein the shaft is attached to the forehead support, wherein the patient interface element comprises a frame part which comprises:
a housing through which the shaft passes;
a drive spring in the housing;
a drive member for driving the shaft having a drive configuration in which it grips the shaft and a release configuration in which it releases the shaft thereby to implement a pump and hold adjustment mechanism, wherein the drive member is biased by the drive spring to the release configuration; and
wherein the locking means comprises a locking member for locking the shaft having a locked configuration in which it grips the shaft and a release configuration in which it releases the shaft, wherein the locking member is biased by a locking spring to the locked configuration.

3. A patient interface as claimed in claim 2, wherein the drive member comprises a pivoted plate having an opening through which the shaft passes and which is biased by the drive spring into an orientation perpendicular to the shaft axis.

4. A patient interface as claimed in claim 2, wherein the locking member comprises a pivoted plate having an opening through which the shaft passes and which is biased by the locking spring into an orientation offset from perpendicular to the shaft axis.

5. (canceled)

6. A patient interface as claimed in claim 1, wherein the shaft is attached to the patient interface, and the opening is formed in a tube attached to the forehead support, wherein the locking means comprises a frictional pad which is coupled to the shaft by a spring mechanism and is biased against the inner surface of the tube, wherein the release button is for depressing the frictional pad against the bias of the spring mechanism.

7. A patient interface as claimed in claim 6, wherein the tube comprises a slot through which the release button extends.

8. A patient interface as claimed in claim 1, further comprising a headgear for holding the patient interface element and forehead support against the head of the patient.

9. A patient interface comprising:
a patient interface element for delivering a breathing gas to a patient; and
a forehead support coupled to the patient interface, wherein the forehead support comprises a forehead cam arrangement and the patient interface element comprises a patient interface cam arrangement, wherein the relative positioning of the patient interface element cam arrangement and the forehead cam arrangements in a first plane implements adjustment of the position of the forehead support along an axis perpendicular to the first plane.

10. A patient interface as claimed in claim 9, comprising an elastic connector which couples the cam arrangements together.

11. A patient interface as claimed in claim 9, wherein the forehead cam arrangement and the patient interface cam arrangement each comprise two side by side cam blocks, wherein the frame cam arrangement blocks are slideable in the first plane relative to the patient interface, wherein the position adjustment is effecting by moving the patient interface cam arrangement blocks together or apart.

12. A patient interface as claimed in claim 9, wherein the cam arrangements each comprise a stepped cam surface to define a set of discrete adjustment locations.

13. A patient interface assembly as claimed in claim 9, further comprising a headgear for holding the patient interface element and forehead support against the head of the patient.

14. A patient interface comprising:
a patient interface element for delivering a breathing gas to a patient; and
a forehead support coupled to the patient interface element, wherein one of the forehead support and the patient interface element comprises a shaft and the other of the forehead support and the patient interface element comprises an opening in which the shaft is received, with the shaft slidable within the opening to permit adjustment of the position of the forehead support relative to the patient interface element, wherein locking means is provided for fixing the position of the shaft and opening, and a release button is provided for releasing the locking means.
